SEQUENCE LISTING (2 sequences)

SEQ ID NO: 1 - DNA, 20 bp, Artificial Sequence
  Naa10p-F
cagcactgcaaccttctctg

SEQ ID NO: 2 - DNA, 20 bp, Artificial Sequence
  Naa10p -R
cacatcgtctgggtcctctt

(19) United States
(12) Patent Application Publication
(10) Pub. No.: US 2022/0339214 A1
(43) Pub. Date: Oct. 27, 2022

(54) KIRIN FRUIT FERMENTATION AND METHODS FOR IMPROVING METABOLISM BY USING THE SAME

(71) Applicant: TCI CO., LTD., Taipei (TW)

(72) Inventors: YUNG-HSIANG LIN, Taipei (TW); PEI-YI WU, Taipei (TW); HUAN-YOU LIN, Taipei (TW)

(21) Appl. No.: 17/709,445

(22) Filed: Mar. 31, 2022

Related U.S. Application Data
(60) Provisional application No. 63/174,548, filed on Apr. 14, 2021.

(51) Int. Cl. A61K 35/744; A23L 33/105; A61P 3/04

(52) U.S. Cl. CPC A61K 35/744; A23L 33/105; A61P 3/04; A61K 2236/19; A61K 2236/331; A61K 2236/51; A61K 2800/85

(57) ABSTRACT

Provided is a method for improving metabolism, including administering to a subject in need thereof a composition including a kirin fruit ferment obtained by fermenting an aqueous extract of a kirin fruit with yeast, lactic acid bacteria, and an acetic acid bacteria, sequentially. The kirin fruit ferment inhibits an expression level of Naa10p gene, increases the activity of mitochondria in beige adipocytes, increases the activity of mitochondria in skeletal muscle cells, promotes the proliferation of skeletal muscle cells, reduces insulin resistance, reduces the content of advanced glycation end products in blood, reduces the content of triglycerides, reduces the arteriosclerosis index, and reduces the liver injury indicator.

### CROSS-REFERENCE TO RELATED APPLICATION

This application claims the benefit of U.S. provisional application Ser. No. 63/174,548, filed on Apr. 14, 2021. The entirety of the above-mentioned patent application is hereby incorporated by reference herein and made a part of the specification.

### REFERENCE OF AN ELECTRONIC SEQUENCE LISTING

The contents of the electronic sequence listing (P211908USI_ST25.txt; Size: 639 bytes; and Date of Creation: Mar. 29, 2022) is herein incorporated by reference in its entirety.

### BACKGROUND

### Technical Field

The present invention relates to a kirin fruit ferment, and in particular, to use of the kirin hint ferment in preparing a composition for improving metabolism.

### Related Art

Kirin fruit (scientific name: Hylocereus megalanthus), also referred to as yellow dragon fruit or yellow pitahaya, is the fruit of a giant cactus “Overlord flower” of the family Cactaceae.

Kirin fruit has a yellow skin outside, a white pulp inside, and black seeds scattered in the pulp. Compared with other dragon fruit varieties, it has a smaller fruit and larger black seeds.

Further, compared with other dragon fruit varieties, the fruit of kirin fruit grows very slowly, with the time from flowering to bearing fruit being about 3 to 5 times that of general dragon fruit, so farmers are less willing to plant it.

The daily routine and diet of modern people different from those in the past cause unbalanced metabolism, also referred to as metabolic syndrome. The metabolic syndrome refers to a clustering of at least three of the following five conditions: excessively large waist circumference, excessively high blood pressure, excessively high blood sugar, excessively high triglycerides, and excessively high high-density cholesterol (HDC). Obese people to develop metabolic syndrome are three times than those having normal weight

### SUMMARY

To further enhance the value of kirin fruit, the inventor continues to research and develop kirin. fruit-related products and their uses.

In view of this, the present invention provides use of a kirm fruit ferment in preparing a composition for improving metabolism, where the kirin fruit ferment is obtained by fermenting an aqueous extract of a kirin fruit with yeast, lactic acid bacteria, and an acetic acid bacteria, sequentially.

In an embodiment, a kirin fruit ferment includes at least myo-inositol, phenyllactic acid, tyrosol, and 4-hydroxyphenyllactic acid. In an embodiment, a kirin fruit ferment includes at least 890 ppm of myo-inositol.

In an embodiment, a kirin fruit ferment inhibits an expression level of Naa10p gene.

In an embodiment, a kirin fruit ferment increases the activity of mitochondria in beige adipocytes. In an embodiment, a kirin fruit ferment increases the activity of mitochondria in skeletal muscle cells.

In an embodiment, a kirin fruit ferment can promote the proliferation of skeletal muscle cells.

In an embodiment, a kirin fruit ferment can reduce insulin resistance.

In an embodiment, a kirin fruit ferment can reduce art least one of the content of advanced glycation end products in blood, the content of triglycerides, the arteriosclerosis index, and the liver injury indicator, or a combination thereof.

In an embodiment, an effective dose of a kirin fruit ferment is 6 mL/day.

Based on the above, the kirin fruit ferment according to any embodiment of the present invention may be used for preparing a composition for improving metabolism. In other words, the composition in an effective dose of 6 mL/day has one or more of the following functions: inhibiting an expression level of Naa10p gene, increasing the activity of mitochondria in beige adipocytes, increasing the activity of mitochondria in skeletal muscle cells, promoting the proliferation of skeletal muscle cells, reduce insulin resistance, reducing the content of advanced glycation end products in blood, reducing the content of triglycerides, reducing the arteriosclerosis index, and reducing the liver injury indicator.

### BRIEF DESCRIPTION OF THE DRAWINGS

FIG. 1 is a graph showing results of promoting the expression of Naa10p gene by a kirin fruit ferment.

FIG. 2 is a graph showing results of the experiment of promoting the activity of mitochondria in skeletal muscle cells by a kirin fruit ferment

FIG. 3 is a graph showing, results of the experiment of promoting the activity of mitochondria in beige adipocytes by a kirin fruit ferment.

FIG. 4 is a graph showing experimental results of the content of advanced glycation end products in the human subject experiment of a kirin fruit ferment.

FIG. 5 is a graph showing experimental results of insulin resistance in the human subject experiment of a kirin fruit ferment.

FIG. 6 is a graph showing experimental results of insulin resistance in the human subject experiment of a kirin fruit ferment.

FIG. 7 is a graph showing experimental results of the content of triglycerides in the human subject experiment of a kirin fruit ferment.

FIG. 8 is a graph showing experimental results of the content of very low-density lipoprotein in the human subject experiment of a kirin fruit ferment.

FIG. 9 is a graph showing experimental results of the arteriosclerosis index in the human subject experiment of a kirin fruit ferment.

FIG. 10 is a graph showing experimental results of the liver injury indicator in the human subject experiment of a kirin fruit ferment.

FIG. 11 is a spectrum of a bioactive substance TCI-GHU-0l in a kirin fruit ferment.

FIG. 12 is a. spectrum of a bioactive substance TCI-GHU-04 in a kirin fruit ferment.

FIG. 13 is a spectrum of a bioactive substance TCI-GHU-03 in a kirin fruit ferment.

FIG. 14 is a spectrum of a bioactive substance TCI-GHU-02 in a kirin fruit ferment.

FIG. 15 is a graph showing experimental results of the total polyphenols content of a kirin fruit ferment.

FIG. 16 is a spectrum of a kirin fruit aqueous extract.

FIG. 17 is a spectrum of a kirin fruit ferment.

FIG. 18 is a graph showing experimental results of promoting the proliferationof skeletal muscle cells by a bioactive substance TCI-GHU-01.

FIG. 19 is a graph showing experimental results of promoting the activity of mitochondria in skeletal muscle cells by a bioactive substance TCI-GHU-01.

### DETAILED DESCRIPTION

As used herein, concentration symbols “%” and “wt %” generally refer to weight percent concentration, and a concentration symbol “vol %” generally refers to volume percent concentration.

As used herein, the “kirin fruit” refers to the fruit of kirin fruit (scientific name: Hylocereus megalanthus).

In some embodiments, a kirin fruit ferment is obtained by fermenting an aqueous extract from a kirin fruit with a yeast, a lactic acid bacterium, and an acetic acid bacterium in sequence.

In some embodiments, kirin fruit is a whole fruit including the skin, pulp, and seeds. In some embodiments, kirin fruit is a whole fruit originating in Peru.

In some embodiments, a whole fruit of kirin fruit may be original, dried, frozen, or processed by other physical methods to facilitate processing, and may further be whole, chopped, diced, milled, ground, or processed by other methods to affect the size and physical integrity of the original material.

In some embodiments, an aqueous extract is extracted from kirin fruit and water in a ratio of 1:15. In some embodiments, an aqueous extract is extracted by mixing and crushing kirin fruit with water. In an embodiment, an aqueous extract is extracted by mixing and crushing kirin fruit with water, and heating to 95° C. for 60 min. In an embodiment, an aqueous extract is extracted by mixing and crushing kirin fruit with water, adding 10% of glucose, and heating to 95±5° C. for 60 min.. In an embodiment, the sugar content of an aqueous extract is greater than or equal to 9.0° Bx,

In some embodiments, a yeast, a lactic acid bacterium, and an acetic acid bacterium are added in sequence into an aqueous extract cooled down for three-stage fermentation to prepare a kirin fruit ferment. In some embodiments, strains are directly added into an aqueous extract for fermentation without filtering, oat solid substances (that is, kirin fruit) therein, so as to further extract active ingredients from the solid substances by using the strains.

In an embodiment, a yeast may be Saccharomyces cerevisiae. For example, the yeast may be Saccharomyces cerevisiae with a deposit number BCRC20271 (an international deposit number ATCC26602) or other commercially available Saccharomyces cerevisiae.

In an embodiment, a lactic acid bacterium may be Lactobacillus plantarum or Lactiplantibacillus plantarum. For example, the lactic acid bacterium may be Lactobacillus plantarum TCI378 with a deposit number BCRT910760 (an international deposit number DSM32451),

In an embodiment, an acetic acid bacterium may be Acetobacter aceti with a deposit number BCRCI1688 (an international deposit number ATCC15973),

In some embodiments, a three-stage fermentation includes adding 0.05-0.15 wt % of yeast into an aqueous extract and standing for fermentation at room temperature for 24 h to form a primary fermentation broth, then adding 0.025-0.01 wt % of lactic acid bacteria and standing for fermentation at room temperature for 24 h to form a secondary fermentation broth, and finally adding 4-6 wt % of lacetic acid bacteria and standing at room temperature to ferment for 120 h to form a plant fermented stock solution. In some embodiments, 0.1 wt % of yeast is added into an aqueous extract and left to ferment at room temperature for 24 h to form a primary fermentation broth, 0.05 wt % of lactic acid bacteria are added and left to ferment at room temperature for 24 h to form a secondary fermentation broth, and 5 wt % of acetic acid bacteria are added and left to ferment at room temperature for 1.20 h to form a plant fermented stock solution.

Herein, the fermentation sequence of yeast, lactic acid bacterium, and acetic acid bacterium cannot be reversed or adjusted. The yeast is first added into the aqueous extract for fermentation to produce alcohol, which helps extract different active ingredients from the kirin fruit. The lactic acid bacterium is then added to further consume glucose in the primary fermentation broth to reduce the sugar content and to produce lactic acid to reduce the pH value. The decrease in pH value helps further extract different active ingredients from the kirin fruit. The acetic acid bacterium is finally added to consume alcohol in the secondary fermentation broth and further reduce the content of glucose.

In some embodiments, after three-stage fermentation, a filtrate is obtained by filtering with a filter. In some embodiments, a filtrate is concentrated under reduced pressure. to obtain a concentrate, which can help remove residual alcohol to ensure that no alcohol remains in the concentrate. Herein, the concentration under reduced pressure is carried out at 55-65° C.

In some embodiments, after concentration under reduced pressure, water is added to adjust a weight back to an original total weight before the concentration under reduced pressure, to obtain an original kirin fruit ferment. In some embodiments, after adjustment by adding water, 60% of isomahooligosaccharides are added to obtain a kirin fruit ferment. In some embodiments, isomaltooligosaccharides are added into an original kirin fruit ferment to a pH value of 3.4±1 and a sugar content of 38±2 ^{°}Bx, to obtain a kirin fruit. ferment.

In some embodiments, a plant fermented stock solution is used as a kirin fruit ferment. In some embodiments, a filtrate is used as a kirin fruit ferment, lin some embodiments, a concentrate is used as a kirin fruit ferment, some embodiments, an original kirin fruit ferment is used as a kirin fruit ferment.

In some embodiments, the present invention provides use of a kirin fruit ferment in preparing a composition for improving metabolism.

In some embodiments, a kirin fruit ferment includes at least myo-inositol, phenyllactic acid, tyrosol, and 4-hydroxyphenyllactic acid. .In some embodiments, a kirin fruit ferment includes at least 890 ppm of myo-inositol.

In some embodiments, a kirin fruit ferment inhibits an expression level of Naa10p gene. Studies show that obesity is related to the overexpression of Naa10p gene, which means that the inhibition of Naa10p enzyme activity in adipose tissue can effectively inhibit overweight, so as to reduce the risk of metabolic syndrome.

In some embodiments, metabolism can he achieved by at least one of increasing the activity of mitochondria in beige adipocytes, increasing the activity of mitochondria in skeletal muscle cells, promoting the proliferation of skeletal muscle cells, reducing insulin resistance, and reducing the content of advanced glycation end products in blood, the content of trialycerides, the arteriosclerosis index, and the liver injury indicator.

In an embodiment, a composition for improving skin condition is a food, drink, or nutritional supplement including a kirm fruit ferment with an effective dose of 6 mL/day. In other words, the food, drink, or nutritional supplement contains a specific content of kirin fruit ferment. In some embodiments, the food may be a general food, food for special health use (FoSHU), dietary supplement, or food additive,

The FoSHU, also referred to as a functional food, refers to a food that is processed to not only supply nutrients but also provide a desirable bioregulatory function. The term “functional” refers to providing nutrients for the structure and functional regulation of the human body or providing a desirable effect for health care purposes such as physiological effects. The food of the present invention can be prepared by a method commonly used in the art, and in the above preparation, it can be prepared by adding raw materials and ingredients commonly added in the art. In addition, the dosage form of the food can be prepared without limitation as long as it is regarded as a dosage form of a food. The food composition of the present invention can be prepared in a variety of dosage forms. Different from ordinary drugs, the food composition using food as a raw material has no side effects that may occur due to long-term drug use, and is easy to carry. Therefore, the food of the present invention can be taken as an auxiliary agent for enhancing the immune enhancement effect.

In some embodiments, the foregoing food may be manufactured into a dosage form suitable for oral administration using techniques well known to those skilled in the art, lin some embodiments, the general food may be, but is not limited to; beverages, fermented foods, bakery products, or condiments.

The composition may further include a physiologically acceptable carrier. The type of carrier is not particularly limited, and any carrier commonly used in the art may be used.

In addition, the composition may contain additional ingredients that are commonly used in foods to improve smell, taste, vision, and the like. For example, the composition may contain 0.1-5 wt % of vitamins A, C, D, E, B1, B2, B6, B12, niacin, biotin, folate, pantothenic acid, etc. In addition, the composition may contain minerals such as zinc (Zn), iron (Fe), calcium (Ca), chromium (Cr), magnesium (Mg), .manganese (Mn), copper (Cu), chromium (Cr), etc. In addition, the composition may contain amino acids such as lysine, tryptophan, cysteine, and valine.

In addition, the composition may contain food additives such as oxidation inhibitors (e.g., butylhydroxyanisole (BRA) and butylhydroxytoluene (BHT)), colorants (e.g., coal tar dye), fragrances (e.g., vanillin, lactones), color couplers (e.g., sodium nitrite and sodium nitrite), preservatives (e.g,, potassium sorbate, sodium benzoate, salicylic acid, and sodium dehydroacetate), bleaching agent (e.g., sodium sulfite), seasonings (e.g., MSG sodium glutamate), sweeteners (e.g., dulcin, cyclamate, saccharin, and sodium), bulking agents (e.g., alum, and D-potassium hydrogen tartrate), fortifiers, emulsifiers, thickeners (paste), filming agents, glue bases, foam inhibitors, solvents, improvers, etc. One or more of the above-mentioned additives can be selected and added in an appropriate amount according to the type of food.

In some embodiments, the kirin fruit ferment (as a food additive) of any embodiment can be added during the preparation of raw materials by conventional methods, or the kirin fruit ferment (as a food additive) of any embodiment is added in the food preparation process to be prepared with any edible material into an edible product for humans and non-human animals to eat.

In some embodiments, the thregoing composition may he a medicament. In other words, the medicament includes an effective dose of kirin fruit ferment.

In some embodiments, the foregoing medicament may be manufactured into a dosage form suitable for enteral or oral administration using techniques well known to those skilled in the art. The dosage form includes, but is not limited to: a tablet, a troche, a lozenge, a pill, a capsule, a dispersible powder or granule, a solution, a suspension, an emulsion, a syrup, an elixir, a slurry, and other similar substances.

In some embodiments, the foregoing medicament may be manufactured into a dosage form suitable for parenteral or topical administration using techniques well known to those skilled in the art. The dosage form includes, but is not limited to, an injection, a sterile powder, an external preparation, and other similar substances. In some embodiments, the medicament may be administered by a parenteral route selected from a group consisting of the following: subcutaneous injection, intraepidermal injection, intradermal injection, and intralesional injection.

In some embodiments, the medicament may further include a pharmaceutically acceptable carrier widely used in drug manufacturing technology. For example, the pharmaceutically acceptable carrier may include one or more of the following reagents: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome, and other similar substances. The selection and quantity of these reagents fall within the scope of professionalism and routine. techniques of those skilled in the art.

In some embodiments, the pharmaceutically acceptable carrier includes a solvent selected from a group consisting of the following: water, normal saline, phosphate buffered saline (PBS), and aqueous solution containing alcohol.

### EXAMPLE 1

### Preparation of Kirin Fruit Ferment

Raw material: The whole fruit of dried kirin fruit scientific name: Hylocereus megalanthus) originating in Peru.

The kirin fruit was mixed and whipped with water in a ratio of 1:15 to obtain an aqueous extract 01, and 1.0% of glucose relative to a total weight of the aqueous extract was added to form a to-be-fermented base solution. Herein, the sugar content of the to-be-fermented base solution is greater than 9° Bx.

The to-be-fermented base solution was heated to 95° C. and maintained at 95° C. for 60 mm to obtain an aqueous extract 02. Then, the aqueous extract 0.2 was cooled down to less than 38° C. for subsequent fermentation.

0.1 wt % of Saccharomyces cerevisiae was added into the aqueous extract 02 and left to stand for 24 h for culture, to form a primary fermentation broth. Herein, the Saccharomyces cerevisiae is Saccharomyces cerevisiae with a deposit number BCRC20271.

Next, 0.05 wt % of Lactobacillus plantarum was added into the primary fermentation broth and left to stand for 24 h for culture, to form a secondary fermentation broth. Herein, the Lactobacillus plantarum is Lactobacillus plantarum with a deposit number BCRC910760.

Then, 5 wt % of Acetobacter aceti was added into the secondary fermentation broth and left to stand for 120 h for fermentation, to form a plant fermented stock solution. Herein, the Acetobacter aceti is Acetobacter aceti with a deposit number BCRC11688. The plant fermented stock solution has a sugar content less than 3° Bx and a pH value of 3.4±1.

The plant fermented stock solution was filtered by a 200-mesh filter to obtain a filtrate. The filtrate was concentrated under reduced pressure 150 bar at 60° C. to obtain a concentrate. Water was added into the concentrate to adjust a weight back to an original total weight before the concentration under reduced pressure, to obtain an original kirin fruit ferment. 60% of isomaltooligosaccharides relative to the original kirin fruit ferment were added to obtain a kirin fruit ferment.

### EXAMPLE 2

### Experiment on Expression Level of Naa10p Gene

Materials:

Experimental cell strain: mouse bone marrow stromal cells (hereinafter referred to as OP9 cells) of the OP9 cell strain (ATCC CRE,2749™) purchased from the American Type Culture Collection (ATCC®).

Culture medium 01 (preadipocyte growth medium): Alpha medium (purchased from Gibco) containing 20% of fetal bovine serum (purchased from Gibco) and 1% of penicillin-streptomycin (purchased from Gibco).

Culture medium 02 (differentiation medium): Alpha medium (purchased from Gibco) containing 20% of fetal bovine serum (purchased from Gibco) and 1% of penicillin-streptomycin (purchased frorn Gibco).

Reagent: RNA extraction reagent kit (purchased from Geneaid, Taiwan, Lot No.FC24015-G), and KAPA CYBR FAST qPCR reagent kit (purchased from KAPA Biosystems).

Reverse transcriptase: SuperScript®III Reverse Transcriptase (Invitrogen, US).

Detection instrument: AB1 StepOnePlus™ Real-Time PCR system (purchased from the Thermo Fisher Scientific, US).

Test process:

First, 8×10⁴ cells were inoculated into a 24-well culture plate containing 500 μL of culture medium 01 per well, and cultured in a carbon dioxide incubator at 37° C. for 7 days, during which the culture medium 01 was replaced every three days. After 7 days, the state of lipid droplets formed in the cells was observed under a microscope to ensure that the cells have completed differentiation. The differentiated cells were divided into three groups: an experimental group, a blank group, and a control group. Each group was repeated for three times, and the average value was used as a result.

Blank group. only a culture medicare was added to culture at 37T for 6 h.

Control group: the aqueous extract 02 (with a concentration of 0 25 vol %) prepared in Example 1 was added to culture at 37° C. for 6 h,

Experimental group: the kirin fruit ferment (with a concentration of 0,25 vo %) prepared in Example 1 was added to culture at 37° C. for 6 h,

After a supernatant of the cells cultured in the experimental woup and the blank group was removed, the cells were washed with 1×DPBS buffer,. and 0.6 mL of RB Buffer (provided in the RNA extraction reagent kit) was added to lyse the cell membranes to form a cell-lysed solution.

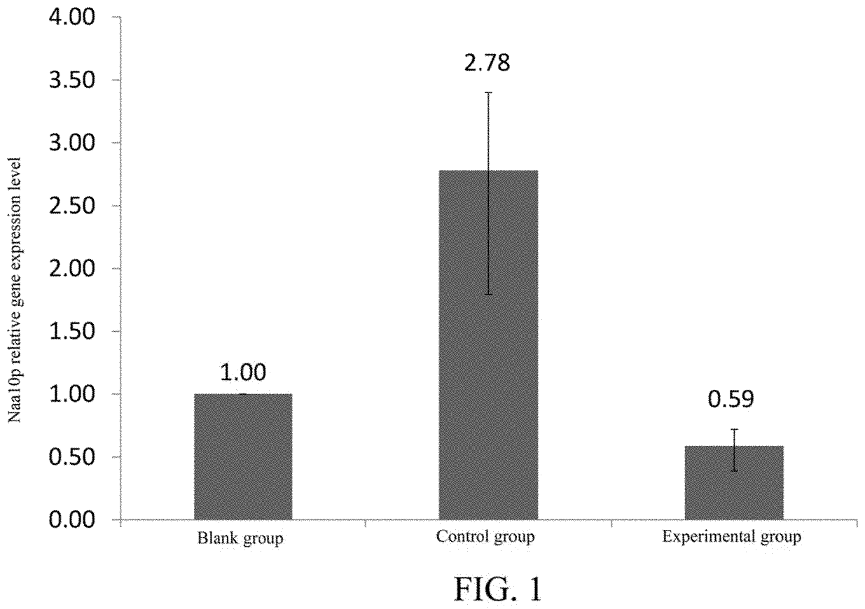

Next, RNA of the three groups of cell lysed solutions was collected separately by using the RNA extraction reagent kit. Then, 1000 ng of the extracted RNA in each group, as a template, was reverse-transcribed with the SuperScript' III reverse transcriptase (purchased from Invitrogene. US) by primer binding to generate corresponding cDNA. Subsequently, the quantitative real-time reverse transcription polymerase chain reaction was carried out on the three groups of reverse-transcribed products respectively with the combination primers in Table 1 by using the ABI StepOnePlus™ Real-Time PCR system (Thermo Fisher Scientific, US) and the KAP-SYBR FAST qPCR Kits to observe the expression level of gene of the blank group, the control group, and the experimental group. The instrument setting conditions for the quantitative real-time reverse transcription polymerase chain reaction were 95° C.. for 1 s, 60° C. for 20 s, a total of 40 cycles, and the relative quantification of gene expression was carried out by the 2-ΔCt method. Herein, as shown in FIG. 1, the quantitative real-time reverse transcription polymerase chain reaction with cDNA can indirectly quantify the mRNA expression level of each gene, and then infer the expression level of the protein encoded by each gene.

TABLE 1

| Target | Primer | Sequence | | |
|---|---|---|---|---|
| gene | name | number | Sequence | Length |
| Naa10p | Naa10p-F | SEQ ID | CAGCACTGCAACCTTCTCTG | 20 |
| | | NO: 1 | | |
| | Naa10p-R | SEQ ID | CACATCGTCTGGGTCCTCTT | 20 |
| | | NO: 2 | | |

Herein, the relative expression level of the target gene was determined by the 2-ΔΔCT method. The relative expression level is defined as a multiple of the RNA expression level of a target gene relative to the corresponding gene in the control group. This method uses the cycle threshold (CT) of the GAPDH gene as the CT of the reference gene of the internal control, and calculates the fold change according to the following formula: ΔCT =CT of target gene in experimental group or control group—CT of internal control ΔΔCT=ΔCT in experimental group—ΔCT in control group Fold change=2-ΔΔCt average.

As shown in FIG. 1. the obtained results were analyzed by student t-test using Excel software to determine whether there is a statistically significant difference between two sample groups. In the figure, “*” represents a p value less than 0.05, “**” represents a p value less than 0.01, and “***” represents a p value less than 0.001, More “*” represents more significant statistical differences from the blank group. “#” represents a p value less than 0.05, “##” represents a p value less than 0.01, and “###” represents a. p value less than 0.001. More “#” represents more significant statistical differences from the control group

Referring to FIG. 1, when the expression level of the Naa10p gene in the blank group was regarded as 1, an expression level of the Naa10p gene in the control group relative to the blank group was 2.78, and an expression level of the Naa10p gene in the experimental group relative to the blank group was 0.59. That is, compared with the blank group, the expression level of the Naa10p gene in the control group was promoted, and the expression level of the Naa10p gene in the experimental group was inhibited.

It can be learned that the kirin fruit ferment effectively inhibited the expression level of the Naa10p gene, thereby reducing the risk of metabolic syndrome. Compared with the aqueous extract, the kirin fruit ferment significantly and effectively inhibited the expression level of the Naa10p gene.

### EXAMPLE 3

### Experiment on Activity of Mitochondria in Skeletal Muscle Cells

Mitochondria are important organelles for cells to carry out oxidative metabolism and provide energy. High activity of mitochondria indicates good cell metabolism efficiency.

Materials:

Experimental cell strain: mouse myoblasts C2C12 (hereinafter referred to as C2C12 cells) of the C2C12 cell strain(ATCC CRL-1772™) purchased from the American Type Culture Collection (ATCC®).

Culture medium: Dui becco's modified Eagle's medium, purchased from Gibco, US, Gat 11965-092. 10% of fetal bovine serum, purchased from Gibco, US, Gat.10437-025 1% of antibiotic, purchased from Gibco, US, Gat. 1240-062.

Reagents: 10×DPBS buffer (purchased from Gibco. Gat. 1 4200-075), trypan blue dead cell dye (purchased from Lonza, Cat. 17-942E), 10×trypsin-EDTA. (purchased from Gibco), and MitoScreen flow cytometry mitochondrial membrane potential detection kit (BD, Cat. BDB551302) including: JC-1 dye and 10×Assay buffer,

Test process:

First, 1×10⁵ cells were inoculated into each well of a 6-well culture plate to culture at 37° C. for 24 h. The cultured cells were divided into three groups: an experimental group, a blank group, and a control group.

Blank group: only a culture medium was added to culture at 37° C. for 24 h.

Control group: the aqueous extract 02 (with a concentration of 0.5 vol %) prepared in Example 1 was added to culture at 37° C. for 24 h.

Experimental group: the kirin fruit ferment (with a concentration of 0.5 vol %) prepared in Example 1 wa.s added to culture at 37° C. for 24 h.

The experimental medium was removed from the culture plate, and the culture plate was washed with 1 mL of 1×PBS for two times. 200 of trypsin was added into each well to react for 5 min in the dark. After the reaction was completed, a cell medium was added to stop the reaction. The cells and cell medium in each well were collected in a 1.5 mL centrifuge tube, and the centrifuge tube containing the cells and cell medium was centrifuged at 400×g for 5 min. After the centrifugation, a supernatant was removed. The cells were washed with 1×DPBS, and then the centrifuge tube containing the cells was centrifuged again at 400×g for 5 min. After the centrifugation again, a supernatant was removed from each centrifuge tube, and 100 μL of JC-1 dye was added into each centrifuge tube and left to stand for 15 min in the dark. After 15 min, each centrifuge tube was centrifuged at 400×g for 5 min. After the centrifugation, a supernatant was removed from each centrifuge tube, and the cells were washed with 1×Assay buffer and centrifuged at 400×g for 5 min. This step was repeated for two times. After the second centrifugation, a supernatant was removed from each centrifuge tube, and the cells in each centrifuge tube were resuspended with 200 μL of 1×DPBS (with 2% of FBS added) to obtain a to-be-tested cell solution. Finally, the fluorescence signal of the to-be-tested cell solution in each well was measured by the flow cytometry (excitation light: 488 nip and scattered light: 527 nm & 590 nm), and the membrane potential of mitochondria was calculated, to analyze the activity of mitochondria.

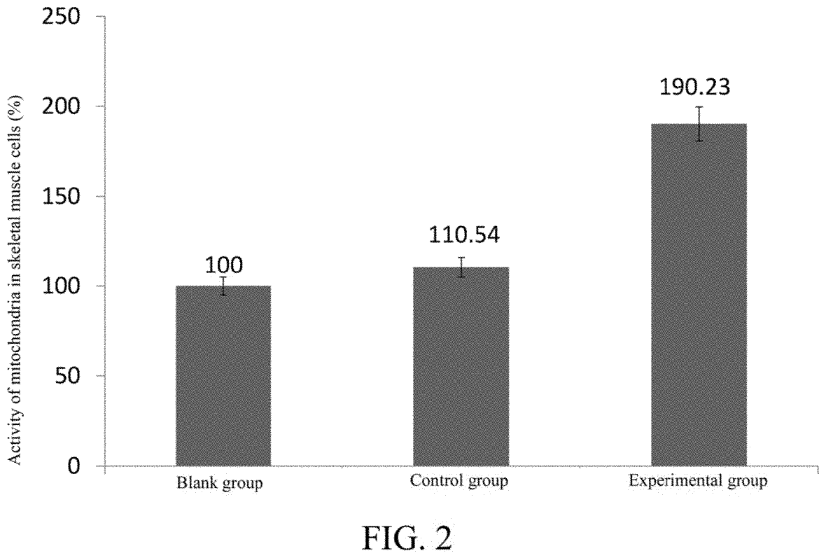

Referring to FIG. 2, when the activity of mitochondria of the blank group was regarded as 100%, the activity of mitochondria of the control group relative to the blank group was 110.54%, and the activity of mitochondria of the experimental group relative to the blank group was 190.23%. Compared with the blank group, a slight increase in the activity of mitochondria can be observed in the control group, but there was no statistically significant difference. It can be further observed that, compared with the blank group or the control group, the activity of mitochondria of the experimental group was significantly increased by more than 80%.

It can be learned that the kirin fruit ferment can effectively increase the activity of mitochondria m skeletal muscle cells, thereby increasing muscle metabolism efficiency.

### EXAMPLE 4

### Experiment on Activity of Mitochondria in Beige Adipocytes

Beige adipocytes contain a high density of mitochondria, which are believed to help convert fat into energy. In this experiment, beige adipocytes were stained to observe the activity of mitochondria therein.

Materials:

Experimental cell strain: mouse bone marrow stromal cells (hereinafter referred to as OP9 cells) of the OP9 cell strain (ATCC CRL-2749™) purchased from the American Type Culture Collection (ATCC®).

Culture medium 01 (preadipocyte growth meth medium): Alpha medium (purchased from Gibco) containing 20% of fetal bovine serum (purchased from Gibco) and 1% of penicillin-streptomycin (purchased from Gibco).

Culture medium 02 (differentiation medium): Alpha medium (purchased from Gibco) containing 20% of fetal bovine serum (purchased from Gibco) and 1% of penicillin-streptomycin (purchased from Gibco).

Reagents: 10×DPBS buffer (purchased from Gibco, Gat.14200-075), trypan blue dead cell dye (purchased from Lonza, Cat.17-942E), 10×trypsin-EDTA (purchased from Gibco), and MitoScreen flow cytometry mitochondrial membrane potential detection kit (BD, Cat BDB551302) including: JC-1 dye and 10×Assay buffer.

Test process:

First, 2×10⁴ cells were inoculated into each well of a 24-well culture plate, and cultured at 37° C. for 1-2 weeks, during which the culture medium 01 was replaced every three days. The formation state of lipid droplets, and generation and aggregation of lipid droplets in the cells were observed under a microscope to ensure that the cells have completed differentiation. The differentiated cells were divided into two groups: an experimental group and a blank group.

Blank group: only a culture medium was added to culture at 37° C. for 24 h.

Experimental group; the kirin fruit ferment (with a concentration of 0.25 vol %) prepared in Example 1 was added to culture at 37° C. for 24 h.

The experimental medium was removed from the culture plate, and the culture plate was washed with 1 of 1×PBS for two times. 200 μL of trypsin was added into each well to react for 5 min. After the reaction was completed, a cell medium was added to stop the reaction. The cells and cell medium in each well were collected in a 1.5 mL centrifuge tube, and the centrifuge tube containing the cells and cell medium was centrifuged at 400×g for 5 min. After the centrifugation, a supernatant was removed. The cells were washed with 1×DPBS, and then the centrifuge tube containing the cells was centrifuged again at 400×g for 5 min. After the centrifugation again, a supernatant was removed from each centrifuge tube, and 100 μL of JC-1 dye was added into each centrifuge tube and left to stand for 15 min in the dark. After 15 min, each centrifuge tube was centrifuged at 400×g for 5 min. After the centrifugation, a supernatant was removed from each centrifuge tube, and the cells were washed with 1×Assay buffer and centrifuged at 400×g for 5 min. This step was repeated for two times. After the second centrifugation, a supernatant was removed from each centrifuge tube, and the cells in each centrifuge tube were resuspended with 200 μL of 1×DPBS (with 2% of FBS added) to obtain a to-be-tested cell solution. Finally, a picture of the fluorescence signal of the to-be-tested cell solution was taken by a camera to observe the activity of mitochondria

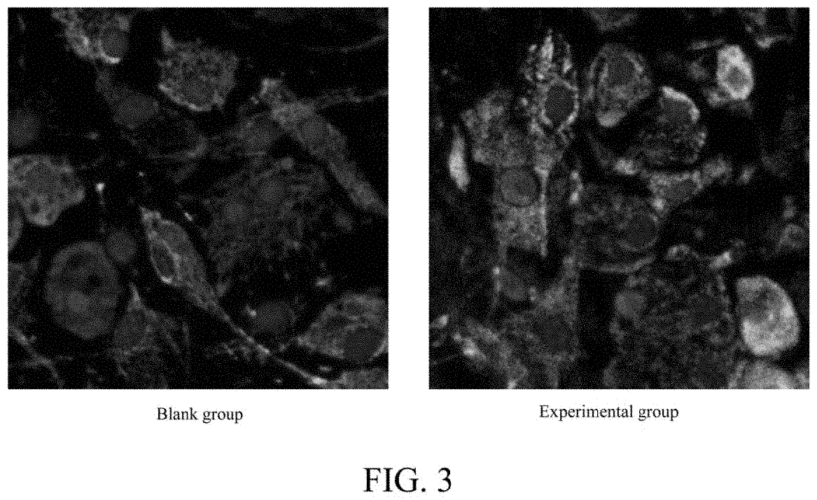

Referring to FIG. 3, in the figure, apparently concentrated circular or oval light spots (blue) were cell nuclei, and small light spots (fluorescent green) without specific shape scattered around the nuclei were active mitochondria. The more small light spots were, the higher the activity of mitochondria was. In the blank group, it was obvious that there were only a few small light spots around the nuclei, indicating that the activity of mitochondria was low. It can be further observed that, compared with the blank group, in the experimental group, there were a large number of small light spots around the nuclei, indicating that the activity of mitochondria was higher than that of the blank group.

It can be learned that the kirin fruit ferment can effectively increase the activity of mitochondria in beige adipocytes, thereby increasing fat metabolism efficiency.

### EXAMPLE 5

### Human Subject Experiment of Knit Fruit Ferment

Subjects: 7subjects (adults aged 25 to 75).

Test items: Blood samples of the subjects were collected ant. entrusted to LEZEN Lab. for test of content of advanced glycation end products (AGES) in blood, insulin resistance, content of triglycerides, content of very low-density lipoprotein (VLDL), arteriosclerosis index, and liver injury indicator ALT.

Test method:

7 subjects were allowed to drink 6 mL of kirin fruit ferment prepared in Example 1 every day for 8 weeks. Blood samples of each subject before drinking (week 0, also referred to as a control group) and after drinking for 8 weeks (week 8, also referred to as an experimental group) were collected for detection.

The statistical significance difference between groups was counted and analyzed through student t-test. In FIG. 5 to FIG. 10, “*” represents a p value less than 0.05, “**” represents a p value less than 0.01, and “***” represents a p value less than 0.001. More “*” represents more significant statistical differences from the control group.

Test result:

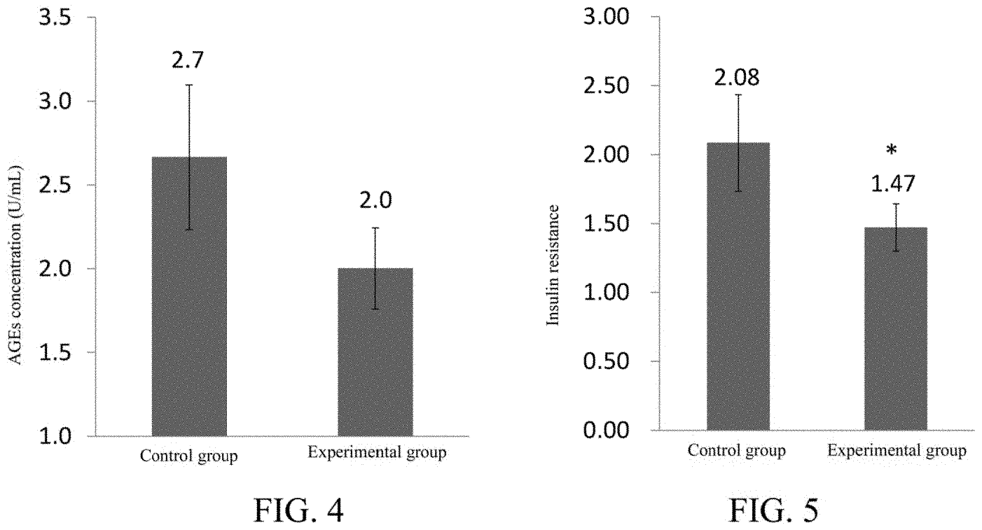

Referring to FIG. 4, after 8 weeks of daily consumption of the kirin fruit ferment, 5 out of 7 subjects had the content of AGES in blood significantly reduced, that is, the proportion of subjects improved was 71.4%. The average content of AGEs in blood of 7 subjects was reduced from 2.7 U/mL to 2.0 U/mL. It indicates that daily consumption of 6 mL or kirin fruit ferment can effectively reduce AGEs in blood, which has a significant anti--glycation effect.

Referring to FIG. 5, after 8 weeks of daily consumption of the kirin fruit ferment, the insulin resistance of 7 subjects was reduced from 2.08 to 1.47. It indicates that daily consumption of 6 mL of kirin fruit ferment can effectively reduce insulin resistance by 29.3%.

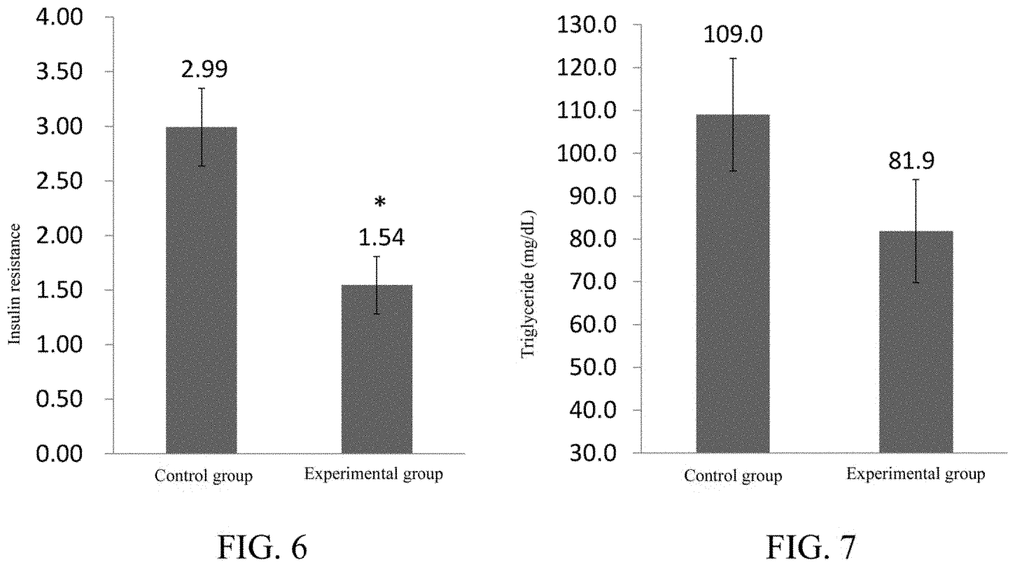

Referrinq to FIG. 6, 3 out of 7 subjects themselves had mild symptoms of insulin resistance, that is, the original insulin resistance of the 3 subjects was greater than 1.9. The cells of the subjects are insensitive to insulin, which prevents glucose in blood from entering the cells. Generally, the higher the insulin resistance is, the higher the risk of diabetes and cardiovascular disease is. After 8 weeks of daily consumption of the kirin fruit ferment, the average insulin resistance of the 3 subjects wars reduced from 2.99 to 1.54. It indicates that daily consumption of 6 mL of kirin fruit ferment can significantly and effectively reduce the insulin resistance of people with high insulin resistance by 48.5%.

Referring to FIG. 7, the average content of triglycerides in blood of 7 subjects was reduced from 109.0 mg/dL to 81.9 mg/dL. It indicates that daily consumption of 6 mL of kirin fruit ferment can effectively reduce the content of triglycerides in blood by 24.9%.

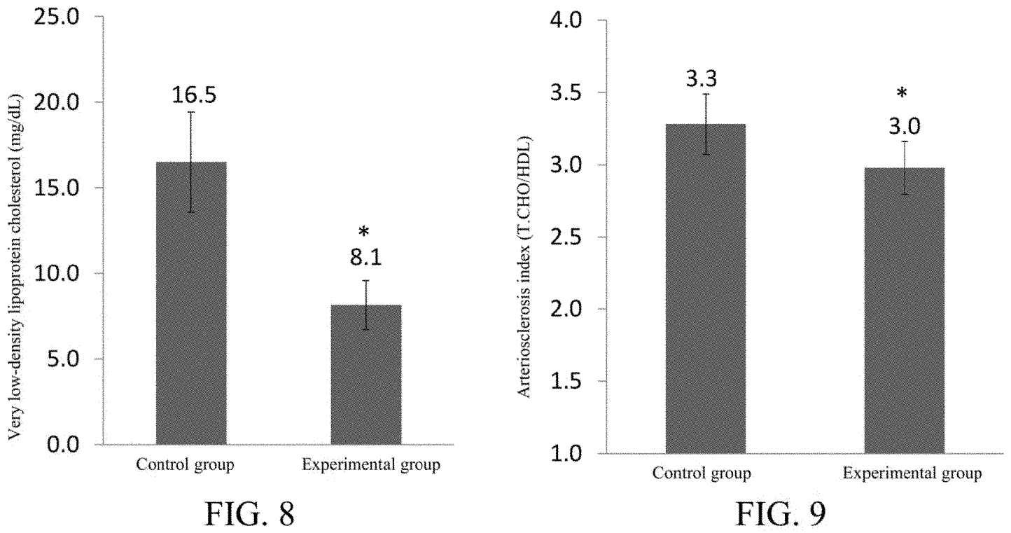

Referring to FIG. 8, the average content of VLDL in blood of 7 subjects was reduced from 16.5 mg/dL to 8.1 mg/dL. It indicates that daily consumption of 6 mL of kirin fruit ferment can effectively reduce the content of VLDL in blood by 50.9%.

Referring to FIG. 9, the average arteriosclerosis index of 7 subjects was reduced from 3.3 TCHO/HDL to 3.0 T.CHO/HDL. It indicates that daily consumption of 6 mL of kirin fruit ferment can effectively reduce arteriosclerosis index by 9.1%.

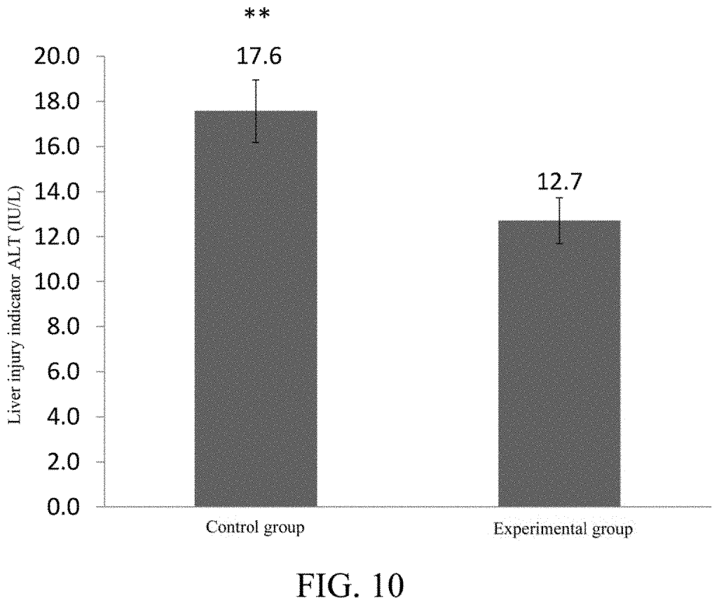

Referring to FIG. 10, the average liver injury indicator of 7 subjects was reduced from 17.6 IU/L to 12.7 IU/L. It indicates that daily consumption of 6 mL of kirin fruit ferment can effectively reduce liver injury indicator by 27.8%, 6 out of 7 subjects have the liver injury indictor significantly reduced, that is, the proportion of subjects improved was 85.7%.

The content of AGEs, insulin resistance, content of triglycerides, content of VLDL, arteriosclerosis index, and liver injury indicator ALT are common index items for determining whether a person is in the risk of metabolic syndrome.

### EXAMPLE 6

### Analysis of Components of Kirin Fruit Ferment

An extract from natural plants usually contains various components and is not a pure substance. Different bioactive substances have different solubilities in different solvents, In this experiment, a specific component in a kirin fruit ferment is transferred frwri a solvent to another solvent immiscible with the solvent.

Instruments and materials:

1. Nuclear magnetic resonance (NMR) spectrometer. 1D and 2D spectra are obtained by using the Ascend 400 MHz spectrometer, Bruker Co., Germany. 15 is used to represent a chemical shift in units of ppm.

2. High resolution liquid chromatography mass spectrometer; Tandem UPLC (Ultimate 3000 HPLC) and high resolution orbital ion trap mass spectrometer (Q-EXACTIVE System with ion Max Source) for determination in units of m/z.

3. Medi um pressure liquid chromatograph (MPLC): CombiFlash® Rf+, Teledyne ISCO, Lincoln, Nebr.

4. High performance liquid chromatograph (HPLC): the HPLC pertains to the Agilent 1200 series, where. a degasser is the Agilent 1322A vacuum degasser; an elution solvent is delivered by using the Agilent G1311 A quaternary pump; a multiple wavelength detector (MWD) is Agilein G1314B; and a diode array detector (DAD) is Agilem 1260 Infinity DAD VL G1315D with detection wavelengths of 210 nm, 280 nm, 320 nm, and 365 nm. (Agilent Germany)

5. Analytical column: Luna® 5 μm C18(2) 100 Å (250×10 mm, Phenomenex, USA).

6. Column chromatography is divided according to packing materials into: column chromatography on the .macroporous Dianion HP-20 resin (Mitsubishi Chemical Co., Japan), normal-phase silica gel column chromatography Merck Kieselgel 60 (40-63 μm, Art. 9385), and reverse-phase silica gel column chromatography Merck LiChroprep® RP-18 (40-63 μm, Art. 0250).

7. Thin-layer chromatography: TLC aluminum sheets (Silica gel 60 F254, 0.25 mm, Merck, Germany) and TLC aluminum sheets (RP-18 F254-S, 0.25 mm, Merck, Germany).

8. UV lamp: UVP UVGL-25 with a wavelength of 254 nm and 365 nm.

9. Solvents and sources thereof: n-butanol, n-hexane, ethyl acetate, acetone, methanol, ethanol, acetonitrile (commercially available from Merck & Co., Taiwan), chloroform-d₁ (with a deateration degree of 99.5%), methanol-d₄ (with a deuteration degree of 99.5%), deuterium oxide (with a deuteration degree of >99.8%), and dimethyl sulfoxide-d6 (with a deutenttion degree of >99.9%) (commercially available from Merck &

Test process:

First, 10 L of kirin fruit ferment was partitioned between n-butanol and water in liquid phase at an equal volume ratio to obtain an extract in n-butanol and a first extract in water. The extract in n-butanol was concentrated under reduced pressure and dried to obtain 21.3 g of extract. from n-butanol fraction (BuF). The first extract in water was concentrated under reduced pressure and dried to obtain 213.5 g of first extract from water fraction (WF).

Next, 100 g of first extract from water fraction was preliminarily separated by column chromatography on the macroporous resin with pure water, pure water-methanol (in an equal volume ratio), and methanol as eluants in sequence. The elution was first carried out with pure water in a flow rate of 30 mL/min for 120 min to obtain a separation fraction WF1, then carried out with pure water-methanol in an equal volume ratio in a flow rate of 30 mL/min for 90 win to obtain a separation traction WF2, and finally carried out with methanol in a flow rate of 30 mlimin for 90 mina separation fraction WF3.

WF1 was separated by using a medium-pressure liquid chromatograph (reverse-phase) and subjected to linear elution from water to methanol in a flow rate of 10 mL/min for 100 min. Subsequently, eluted substances with similar results were combined through thin-layer chromatography to obtain 5 sub-separation fractions: WF1-1 WF1 -2, WF1-3, WF1-4, and WF1-5.

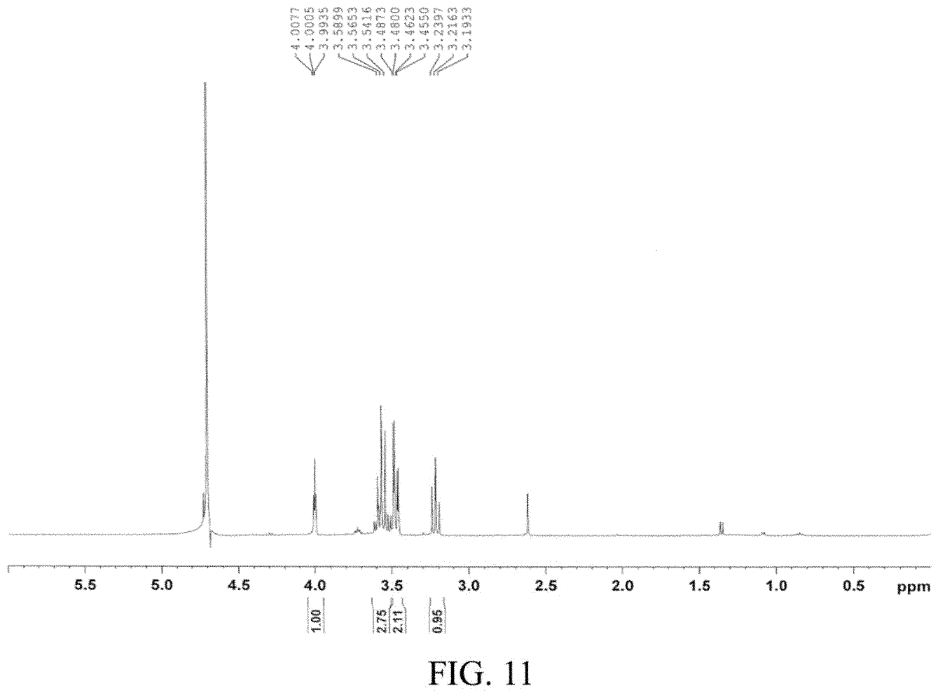

WF1-1was purified through normal-phase silica, gel column chromatography (ethyl acetate/methanol=1/1) to obtain a bioactive substance TCI-GHU-01 The bioactive substance TCI-GHU-01 was analyzed and identified through ¹H-NMR to obtain a spectrum of the bioactive substance TCI-GHU-01, as shown in FIG. 11. After its chemical structure was analyzed, the bioactive substance TCI-GHU-01 was determined as myo-inositol with a structure shown as follows:

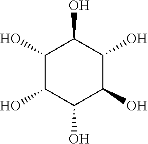 [SMILES: O[C@H]1[C@H](O)[C@@H](O)[C@H](O)[C@@H](O)[C@H]1O]

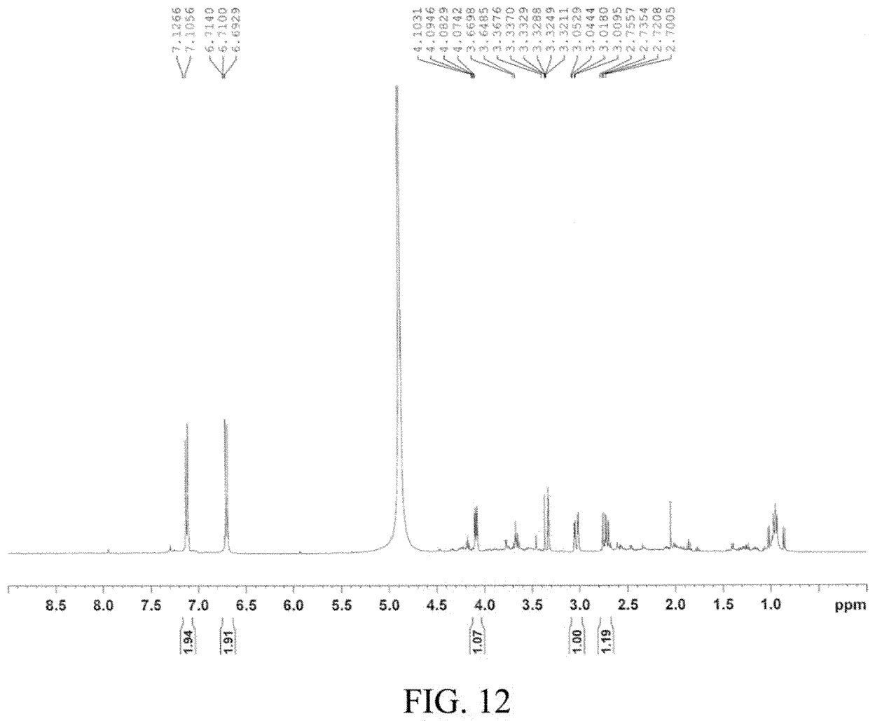

WF1-2 was purified by a medium-pressure liquid chromatograph (reverse-phase) (methanol/water 3/17) to obtain a bioactive substance TCI-GHU-04. The bioactive substance TCI-GHU-04 was analyzed and identified through ¹H-NMR to obtain a spectrum of the bioactive substance TCI-GHU-04, as shown in FIG. 12. After its chemical structure was analyzed, the bioactive substance TC1-GHU-04 was determined as 4-hydroxy-3-phenyllactic acid with a structure shown as follows:

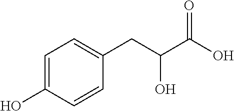 [SMILES: O=C(O)C(O)Cc1ccc(O)cc1]

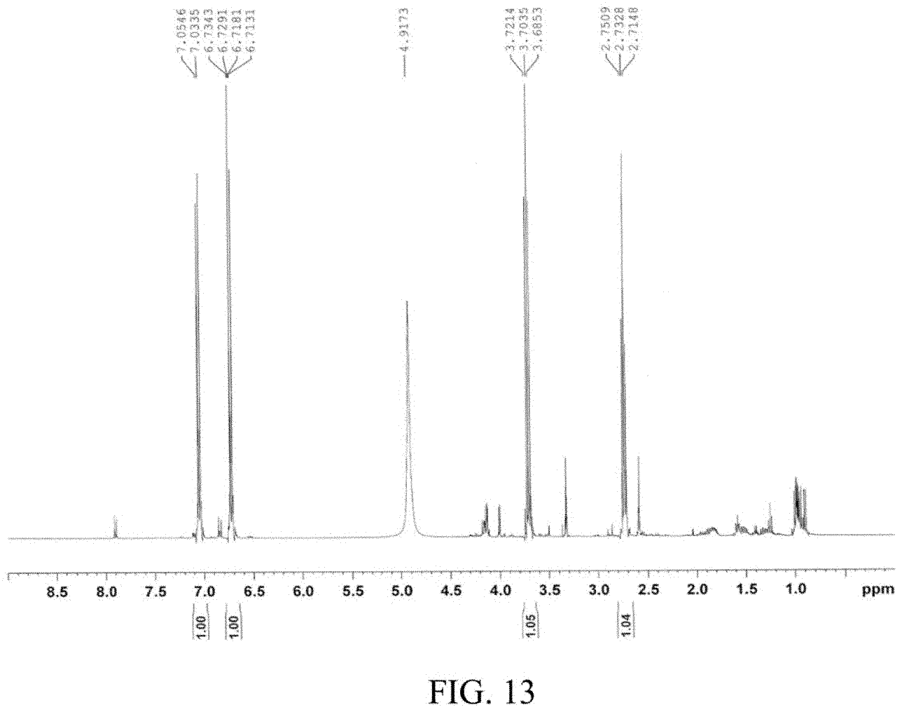

WF1 -3 was purified by a medium-pressure liquid chromatograph (reverse-phase) (methanol. water ⁻1/4) to obtain a bioactive substance TCI-GHU-03. The bioactive substance TCI-GHU-03 was analyzed and identified through ¹H-NMR to obtain a spectrum of the bioactive substance TCI-GHU-03, as shown in FIG. 13. After its chemical structure was analyzed, the bioactive substance TCI-GHU-03 was determined as tyrosol with a structure shown as follows:

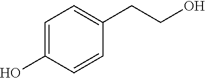

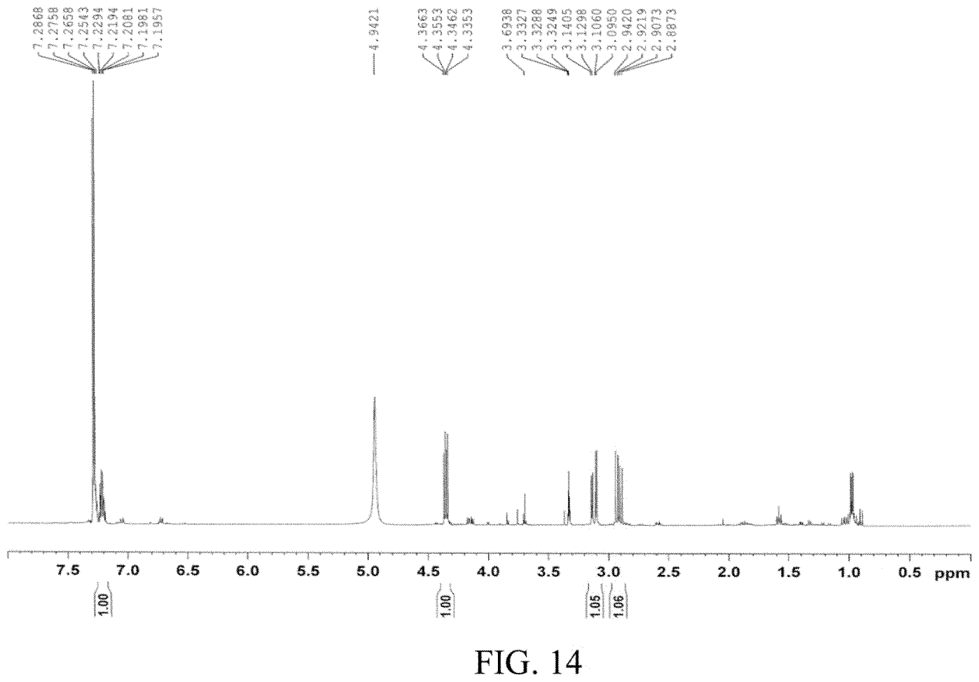

WF1-4 was purified through reverse-phase HPLC (methanol/water=3/7) obtain a bioactive substance TCI-GHU-02. The bioactive substance TCI-GHI-02 was analyzed and identified through ¹H-NMR to obtain a spectrum of the bioactive substance TCI-GHU-02, as shown in FIG. 14. After its chemical structure was analyzed, the bioactive substance TCI-GHU-02 was determined as 3-phenyllactic acid with a structure shown as follows:

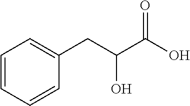 [SMILES: O=C(O)C(O)Cc1ccccc1]

### EXAMPLE 7

### Experiment on Total Polyphenols Content of Kinin Fruit Ferment

Materials: Folin-Ciocalteu's phenol reagent (purchased from Merck, material code: 1.09001.0100), gallic acid (purchased from Signa, material code: G7384), and sodium carbonate anhydrous (purchased from Sigma, material code: 31432).

Test process: 10.0 mg of gallic acid was weighed into a 10 mL volumetric flask and precisely adjusted in volume with water to 10 mL, to obtain a gallic acid stock solution. The gallic acid stock solution was diluted 10-fold, that is, 100 μL of gallic acid stock solution was added into 900 μL of water to obtain a 100 μg/mL initial solution of gallic acid (that is, containing 1000 ppm of gallic acid). Then, 0 μg/mL, 20 μg/mL, 40 μg/mL, 60 μg/mL, 80 μg/mL, and 100 μg/mL standard solutions of gallic acid were prepared according to the following table 2, and 100 μL of each concentration of standard solution was taken into a glass test tube 500 μL of Folin-Ciocalteu's phenol reagent was added in each glass test tube and mixed with the standard solution uniformly, standing for 3 min, and 400 μL of 7.5% sodium carbonate was added to mix uniformly and react for 30 min, to obtain a standard reaction solution 200 μL of the standard reaction solution was taken in a 96-well plate and the absorbance of the standard reaction solution was measured at 750 nm to obtain a standard curve.

TABLE 2

| | Standard concentration | | | | | |
|---|---|---|---|---|---|---|
| | (μg/mL) | | | | | |
| | 0 | 20 | 40 | 60 | 80 | 100 |
| Initial solution | 0 μL | 20 μL | 40 μL | 60 μL | 80 μL | 100 μL |
| Water | 100 μL | 80 μL | 60 μL | 40 μL | 20 μL | 0 μL |

To-be-tested samples were prepared. A sample of an experimental group is the kirin fruit ferment prepared in Example 1. A sample of a control group 01 is the aqueous extract 02 prepared in Example 1. A sample of a control group 02 is an aqueous extract prepared from white dragon fruit (scientific name: Hylocereus undatus) as a raw material by a method, of preparing the aqueous extract 02 in Example A sample of a control group 03 is an aqueous extract prepared from red dragon fruit (scientific name: Hylocereas polyrhizus) as a raw material by a method of preparing the aqueous extract 02 in Example 1.

The sample in each group was diluted 20-fold with water and 100 μL thereof was transferred into a glass test tube. Then, 500 μL of Folin-Ciocalteu's phenol reagent was added in the glass test tube and mixed with the sample uniformly, standing for 3 min, and 400 μL of 7.5% sodium carbonate was added to mix uniformly and react for 30 min, to obtain a to-be-tested reaction solution. The glass test tube containing the to-be-tested reaction solution was shaken to ensure no air bubbles. 200 μL of to-be-tested reaction solution was transferred into a 96-well plate, and an absorbance of the to-be-tested reaction solution at 750 nm was measured. Then, the absorbance of the to-be-tested reaction solution corresponding to each sample was first divided by the sugar content of the sample and then converted into the total polyphenols content based on a standard curve by an interpolation method. The foregoing experimental steps were repeated for three times.

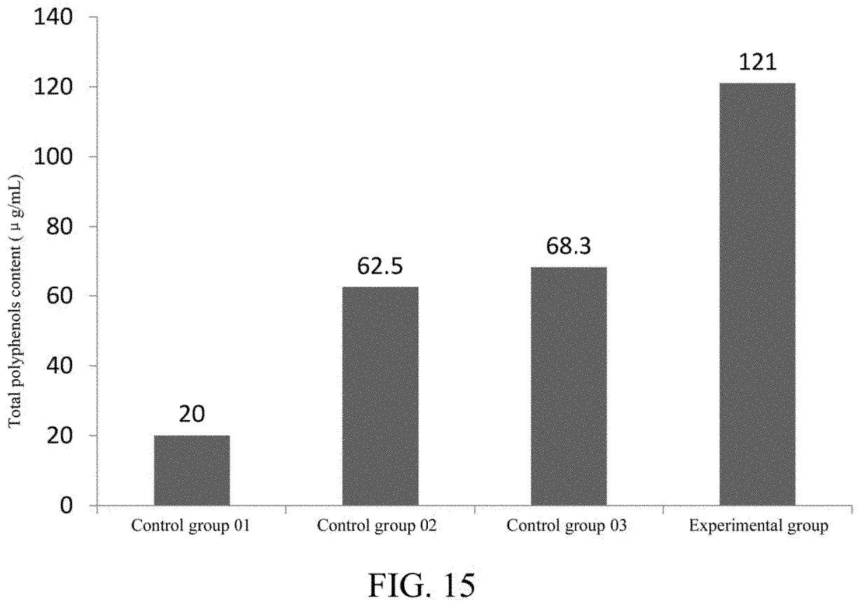

As shown in FIG. 15, the total polyphenols content of the control group 01 was the total polyphenols content of the control group 0.2 was 62.5 μg/mL, the total polyphenols content of the control group 03 was 68.3 μg/mL, and the total polyphenols content of the experimental group was 121 μg/mL. Compared with all the control groups, the total polyphenols content of the experimental group was significantly increased. Especially, compared with the control group 01, which was unfermented, the total polyphenols content of the experimental group was increased by six folds. Herein, it can be inferred that the kirin fruit ferment can release a large amount of total polyphenols after microbial fermentation, and can enhance the antioxidant activity, which can effectively reduce accumulation of free radicals and reduce inflammation.

### EXAMPLE 8

### Analytical Experimentof Kirin Fruit Ferment and Aqueous Extract

Herein, quantitative and qualitative analysis was carried out on biactive substances in the kirin fruit ferment prepared in Example 1 and the aqueous extract 02 prepared in Example 1 by high performance liquid chromatography (HPLC).

Test process:

The solvents used were methanol and water with 0.1% formic acid added each, with a flow rate set to 1 mL/min and an elution condition set to methanol:water of 2:98 at 0 min, methanol:water of 2:98 at 10 min, methanol:water of 70:30 at 40 min, methanol:water of 100:0 at 50 min, and methanol:water of 100:0 at 60 min.

Test result:

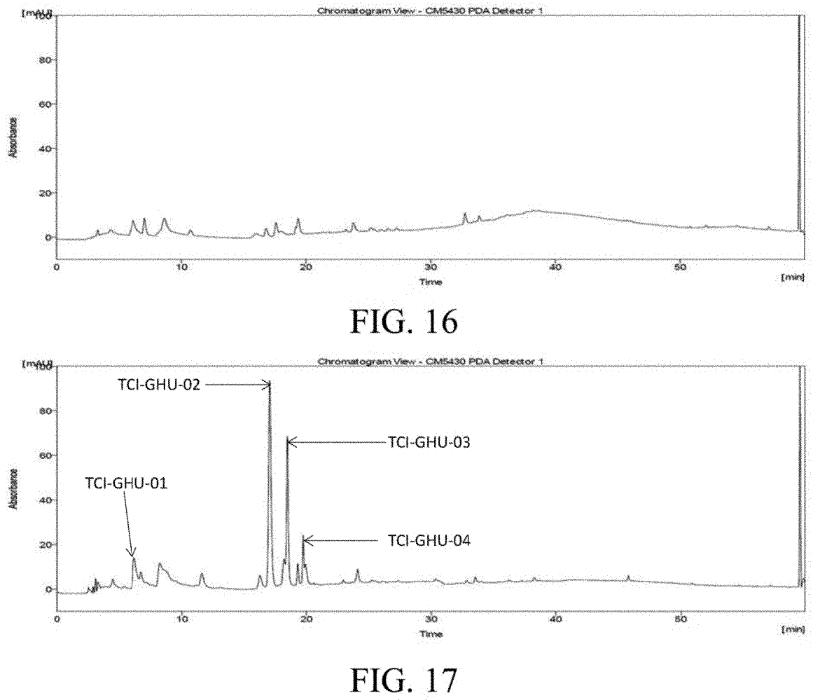

Refer to FIG. 16 and FIG. 17. FIG. 16 is a spectrum of the aqueous extract. FIG. 17 is a spectrum of the kiwi fruit ferment. in FIG. 17, the peak of the bioactive substance TCI-GHU-01 was resolved at about 16 min, and the peaks of the bioactive substances TCI-GHU-04, TCI-GHU-03, and TCI-GHU-02 were resolved in sequence at about 18-20 min.

However, the corresponding peaks are not shown in FIG. 16. In other words, the kirin fruit ferment obtained through three-stage fermentation is changed in components and proportions from the aqueous extract.

### EXAMPLE 9

### Experiment on Proliferation of Skeletal Muscle Cells

People with more muscle tissue have a higher basal metabolic rate. Generally, the basal metabolic capacity of 1 kg of muscle is 13 kcal. That is, if the muscle increases by 1 kg, the calorie consumption will increase by 13 kcal.

Materials:

Experimental cell strain: mouse myoblasts C2C12 (hereinafter referred to as C2C12 cells) of the C2C12 cell strain (ATCC CRL-1772™) purchased from the American Type Culture Collection (ATCC®).

Culture medium: Dulbecco's modified Eagle's medium, purchased from Gibco, US, Gat.11965-092. 10% of fetal bovine serum (FBS), purchased from Gibco, US, Gat.10437-028. 1% of antibiotic, purchased from Gibco, US, Gat.15240-062.

Reagents: 10×DPBS buffer (purchased from Gibco, Gat 14200-075), trypan blue dead cell dye (purchased from Lonza, Cat 17-942E), trypsin-EDTA (purchased from Gibco, Cat. 15400-054), and cell proliferation Click-iT™ Plus EdU kit (Invitrogen; Cat. C10632).

First, 1×10⁵ cells were inoculated into each well of a 6-well culture plate to culture at 37° C. for 24 h. The cultured cells were divided into three groups: an experimental group, a blank group, and a control group.

Blank group: only a culture medium was added to culture at 37° C. for 24 h.

Control group: 10% of PBS was added to culture at 37° C. for 24 h, which was used as a positive control group.

Experimental group: 100 nM of the bioactive substance TCI-GHU-01 obtained in Example 6 was added to culture at 37° C. for 24 h.

Next, the specific reagent EdU from the cell proliferation kit was added to culture for 2 h. Then, the culture medium was removed, the cells were obtained with 0.5% of trypsin in a centrifuge tube and washed with 1×DPBS buffer, and the centrifuge tube containing the cells was centrifuged at 400×g, for 5 min. After the centrifugation, a supernatant was removed from each centrifuge tube, and 100 μL of working reagent (Click-iT™Fixative(Component D)) from the cell proliferation kit was added into each centrifuge tube and left to stand for 15 min M the dark. After 15 min, the cells were washed with 1×DPBS buffer and centrifuged again at 400×g for 5 min. After the centrifugation, a supernatant was removed from each centrifuge tube, and 100 uL of working reagent (Click-iT™ Saponin-based permeabilization) from the cell proliferation kit was added into each centrifuge tube and left to stand for 15 min in the dark. 0.5 mL of reaction reagent from the cell proliferation kit was added into each centrifuge tube and left to stand for 30 min in the dark. The cells were washed with the working reagent (Click-iT™ Saponin-based permeabilization) from the cell proliferation kit, a supernatant was removed, and the cells were dissolved back with 500 μL of working reagent, to obtain a to-be-tested cell solution. Finally, the fluorescence signal of the to-be-tested cell solution in each well was measured by the flow cytometry (under 488 nm of excitation light and 530/30 nm of scattered light), and the quantity of cells was calculated.

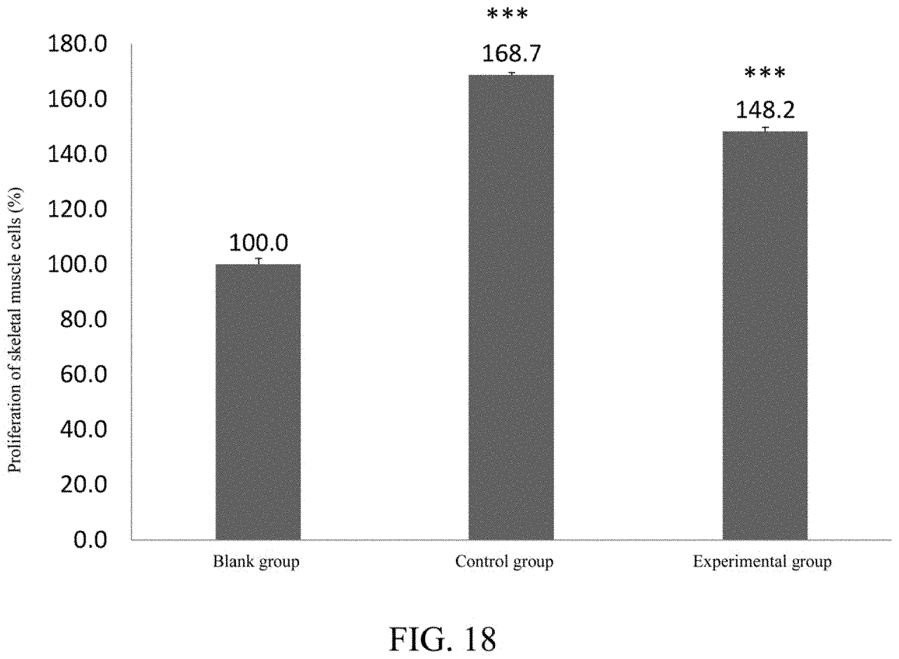

As shown in FIG. 18, the obtained results were analyzed by student t-test using Excel software to determine whether there is a statistically significant difference between two sample groups. In the figure, “*” represents a p value less than 0.05, “**” represents a p value less than 0.01, and “***” represents a p value less than 0.001. More “*” represents more significant statistical differences from the blank group.

Referring to FIG. 18, when the quantity of skeletal muscle cells determined in the blank group was regarded as 100%, the quantity of skeletal muscle cells determined in the control group relative to the blank group was 168.7%, and the quantity of skeletal muscle cells determined in the experimental group relative to the blank group was 148.2%. Compared with the blank group, the experimental group had a statistically significant difference. The quantity of skeletal muscle cells determined in the experimental group was significantly increased by 48.2%.

It can be learned that the kirin fruit ferment can effectively promote the growth of skeletal muscle cells, thereby increasing individual metabolism efficiency.

### EXAMPLE 10

### Experiment on Activity of Mitochondria in Skeletal Muscle Cells Affected by Active Substances

The higher the efficacy of muscle tissue is, the higher the basal metabolic rate is. In this experiment, skeletal muscle cells were stained to observe the activity of mitochondria therein.

Materials: The same as in Example 3 above.

Test process:

First, 1×10⁵ cells were inoculated into each well of a 6-well culture plate to culture at 37° C. for 24 h. The cultured cells were divided into two groups: an experimental group and a blank group.

Blank group: only a culture medium was added to culture at 37° C. for 24 h,

Experimental group: 100 nM of the bioa.ctive substance TCI-GHU-01 obtained in Example 6 was added to culture at 37° C. for 24 h.

The subsequent steps were the same as in Example 3 above. Finally, a picture of the fluorescence signal of the to-be-tested cell solution was taken by a. camera to observe the activity of mitochondria.

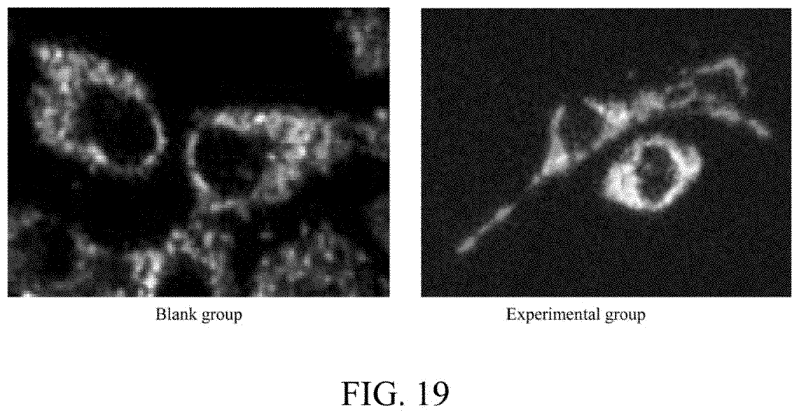

Referring to FIG. 19, in the figure, apparently concentrated circular or oval dark spots were cell nuclei, and small light spots (fluorescent red or green) without specific shape scattered around the nuclei were active mitochondria, The denser small light spots are, the higher the activity of mitochondria is in the blank group, it was obvious that there were only a few small light spots around the nuclei, indicating that the activity of mitochondria was low. It can be further observed that, compared with the blank group, in the experimental group, there were a large number of small light spots around the nuclei, indicating that the activity of mitochondria was higher than that of the blank group.

Based on the above, the kirin fruit ferment according to any embodiment of the present invention may be used for preparing a composition for improving skin conditions. in other words, the composition in an effective dose of 6 mL/day has one or more of the following functions: inhibiting an expression level of Naa10p gene, increasing the activity of mitochondria in beige adipocytes, increasingthe activity of mitochondria in skeletal muscle cells, promoting the proliferation of skeletal muscle cells, reduce insulin resistance, reducing the content of advanced glycation end products in blood, reducing the content of triglycerides, reducing the arteriosclerosis index, and reducing the liver injury indicator.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A method for improving metabolism, comprising administering to a subject in need thereof a composition comprising. a kirin fruit ferment obtained by fermenting an aqueous extract of a kirin fruit (Hylocereus megalanthus) with yeast, lactic acid bacteria, and an acetic acid bacteria, sequentially.

2. The method according to claim 1, wherein the kirin fruit ferment comprises at least myo-inositol, phenyllactic acid, tyrosol, and 4-hydroxyphenyllactic acid.

3. The method according to claim 2, wherein the kirin fruit ferment comprises at least 890 ppm of the myo-inositol.

4. The method according to claim 1, wherein the kirin fruit ferment inhibits an expression level of Naa10p gene.

5. The method according to claim 1, wherein the kirin fruit ferment increases the activity of mitochondria in beige adipocytes.

6. The method according to claim 1, wherein the kirin fruit ferment increases the activity of mitochondria in skeletal muscle cells.

7. The method according to claim 1, wherein the kirin fruit ferment promotes the proliferation of skeletal muscle cells.

8. The method according to claim 1, wherein the kirin fruit ferment reduces insulin resistance.

9. The method according. to claim 8, wherein the kirin fruit ferment reduces at least one of the content of advanced glycation end products in blood, the content of triglycerides, the arteriosclerosis index, and the liver injury indicator, or a combination thereof.

10. The method according to claim 8, wherein an effective dose of the kirin fruit ferment is 6 mL/day.

11. The method according to claim 9, wherein an effective dose of the kirin fruit ferment is 6 mL/day.
